(19) United States
(12) Patent Application Publication
(10) Pub. No.: US 2026/0174413 A1
(43) Pub. Date: Jun. 25, 2026

(54) ULTRASOUND ABLATION OF NERVES

(71) Applicant: RENALY LTD, Yokneam (IL)

(72) Inventors: Ran SELA, Ramat Hasharon (IL); Assaf Rubissa, Yaad (IL); Yuri Megel, Nesher (IL); Guy Ben-Ezra, Karkur (IL); Shai Fisher, Haifa (IL); Urit Gordon, Alonei Abba (IL)

(21) Appl. No.: 19/539,449

(22) Filed: Feb. 13, 2026

Related U.S. Application Data
(63) Continuation of application No. PCT/IB2024/058022, filed on Aug. 18, 2024.
(60) Provisional application No. 63/533,711, filed on Aug. 21, 2023.

(51) Int. Cl. A61B 8/00; A61B 8/06; A61B 8/12

(52) U.S. Cl. CPC A61B 8/445; A61B 8/06; A61B 8/12; A61B 8/4461; A61B 8/483

(57) ABSTRACT

An apparatus (22) includes a tube (30) configured for insertion into a blood vessel of a patient, a transducer housing (32) coupled distally to the tube, a housing cover (28) configured to enclose the transducer housing within the blood vessel, and at least one ablating ultrasound transducer (34) disposed within the transducer housing and configured to emit ablative ultrasound waves, through the housing cover, at one or more nerves (90) in a vicinity of the blood vessel. Other embodiments are also described.

### CROSS-REFERENCE TO RELATED APPLICATIONS

The present disclosure claims the priority of a Continuation Application of PCT Application No. PCT/IB2024/058022, filed on Aug. 18, 2024, which claims the priority of US Provisional Application 63/533,711, entitled “Ultrasound ablation of nerves,” filed Aug. 21, 2023, whose disclosure is incorporated herein by reference.

### TECHNICAL FIELD

The present disclosure is related generally to ultrasound ablation therapy, and particularly to ultrasound ablation of nerves, such as renal nerves.

### BACKGROUND

Resistant hypertension, i.e., hypertension that has not responded to medication, is a significant health problem.

Recently, percutaneous renal denervation has become the treatment of choice for resistant hypertension. In this procedure, an ablative medium, such as radiofrequency energy or ultrasound, is used to ablate the renal nerves from within the renal artery. The resultant reduction in renal-nerve activity leads to a decrease in blood pressure.

### SUMMARY

Some embodiments of the present disclosure provide an apparatus configured to deliver ablative ultrasound waves to nerves surrounding a blood vessel, such as a renal artery, of a patient. The apparatus includes a tube configured for insertion into the blood vessel, a transducer housing coupled distally to the tube, a housing cover configured to enclose the transducer housing within the blood vessel, and at least one ablating ultrasound transducer disposed within the transducer housing and configured to emit ablative ultrasound waves, through the housing cover, at one or more nerves in a vicinity of the blood vessel.

Typically, it is important to carefully choose the locations at which, and the directions in which, the ablative waves are emitted, to account for “nontarget” anatomical structures, such as lymph nodes and other blood vessels, near the blood vessel. Likewise, due to the presence of such structures, it may be necessary to carefully control the amount of delivered ablation energy. A procedure in which the ablative ultrasound is emitted uniformly, in all directions, across the length of the blood vessel, may be ineffective or even dangerous.

To address these challenges, in some embodiments, the apparatus further includes at least one imaging ultrasound transducer disposed within the transducer housing. The imaging ultrasound transducer is configured to emit imaging ultrasound waves at the vicinity of the blood vessel and to transduce reflections of the imaging ultrasound waves. Advantageously, the transduced reflections may facilitate construction of an ultrasound image for guiding the emission of the ablative ultrasound waves, such that the ablation may be performed more effectively and/or safely. For example, a computer processor may construct an ultrasound image based on the transduced reflections, and then process the image so as to identify one or more anatomical structures—such as nerves and/or nontarget structures—in the vicinity of the blood vessel.

In some embodiments, the ablating ultrasound transducer includes an electroacoustic transducing element and one or more flexible elements, such as adhesive strips, supporting the electroacoustic transducing element. Advantageously, the flexible elements may facilitate the vibration of the electroacoustic transducing element.

In some embodiments, the apparatus further includes a fluid conduit disposed within the transducer housing at a first side of the ablating ultrasound transducer and configured to deliver a fluid to the distal end of the transducer housing such that the fluid flows proximally, from the distal end of the transducer housing, over the ablating ultrasound transducer at a second side of the ablating ultrasound transducer, from which the ablative waves are emitted. Thus, advantageously, the fluid may carry away any air that might otherwise impede the ablative waves.

There is therefore provided, in accordance with some embodiments of the present disclosure, an apparatus including a tube configured for insertion into a blood vessel of a patient, a transducer housing coupled distally to the tube, a housing cover configured to enclose the transducer housing within the blood vessel, and at least one ablating ultrasound transducer disposed within the transducer housing and configured to emit ablative ultrasound waves, through the housing cover, at one or more nerves in a vicinity of the blood vessel.

In some embodiments, the transducer housing does not completely enclose the ablating ultrasound transducer.

In some embodiments, the tube is configured to transfer roll torque and linear force to the transducer housing.

In some embodiments, the apparatus further includes a rapid exchange tip coupled distally to the housing cover and configured for passage of a guidewire therethrough.

In some embodiments, the apparatus further includes:
a control handle configured to couple proximally to the tube and including a gear system; and
a motor unit configured to couple to the gear system and to supply mechanical forces to the gear system so as to move the tube.

In some embodiments, the transducer housing is configured to restrict an angular range of the ablative ultrasound waves, such that the ablative ultrasound waves do not ablate around a full circumference of the blood vessel simultaneously.

In some embodiments, a diameter of the housing cover is less than 10 Fr.

In some embodiments, the apparatus further includes a catheter shaft, which contains the tube and is coupled proximally to the housing cover.

In some embodiments, the apparatus further includes an expandable element coupled to an outer wall of the catheter shaft or of the housing cover and configured to expand within the blood vessel, thereby inhibiting contact between the outer wall and tissue of the blood vessel.

In some embodiments, the housing cover has a lower acoustic impedance than does the catheter shaft.

In some embodiments, the ablating ultrasound transducer includes:
an electroacoustic transducing element; and
one or more flexible elements supporting the electroacoustic transducing element.

In some embodiments, the flexible elements include one or more flexible adhesive strips.

In some embodiments, the flexible adhesive strips cover a perimeter of the electroacoustic transducing element.

In some embodiments, the housing cover is coupled distally to the tube.

In some embodiments, the apparatus further includes:
a catheter shaft, which contains the tube; and
an expandable element coupled to an outer wall of the catheter shaft or of the housing cover and configured to expand within the blood vessel, thereby inhibiting contact between the outer wall and tissue of the blood vessel.

In some embodiments, the transducer housing includes a distal aligning element configured to align a guidewire, which passes through the transducer housing, with a roll axis of the transducer housing, such that the guidewire is aligned with the roll axis distally to the transducer housing.

In some embodiments, the aligning element is shaped to define a passageway that is at least partly oblique with respect to the roll axis, and the aligning element is configured to align the guidewire by virtue of the guidewire passing through the passageway.

In some embodiments, the apparatus further includes a guidewire conduit passing through the transducer housing, the guidewire passes through the guidewire conduit, and the aligning element aligns the guidewire with the roll axis by virtue of holding the guidewire conduit in alignment with the roll axis.

In some embodiments, the apparatus further includes:
a cover surrounding at least part of the aligning element; and
a fluid seal disposed between the cover and the aligning element or distally to the aligning element within the cover.

In some embodiments, the transducer housing contains an air pocket on a first side of the ablating ultrasound transducer, such that the ablative ultrasound waves are concentrated at a second side of the ablative ultrasound transducer, which is opposite, and fluidly isolated from, the first side.

In some embodiments, the ablating ultrasound transducer includes:
an electroacoustic transducing element; and
one or more flexible elements that support the electroacoustic transducing element and isolate the air pocket from the second side of ablative ultrasound transducer.

In some embodiments, the apparatus further includes a fluid conduit disposed within the transducer housing at the first side of the ablating ultrasound transducer and configured to deliver a fluid to a distal end of the transducer housing such that the fluid flows proximally, from the distal end of the transducer housing, over the ablating ultrasound transducer at the second side of the ablating ultrasound transducer.

In some embodiments, the apparatus further includes a catheter shaft, which contains the tube, and
the fluid conduit is configured to deliver the fluid such that the fluid flows proximally over the ablating ultrasound transducer and into a space between the tube and the catheter shaft.

In some embodiments, the fluid flows proximally through the housing cover, and the housing cover is inflatable, such that the fluid inflates the housing cover.

In some embodiments, the apparatus further includes at least one imaging ultrasound transducer disposed within the transducer housing and configured to:
emit imaging ultrasound waves, through the housing cover, at the vicinity, and
transduce reflections of the imaging ultrasound waves so as to facilitate construction of an ultrasound image for guiding the emission of the ablative ultrasound waves.

In some embodiments, the imaging ultrasound transducer is distal to the ablating ultrasound transducer.

In some embodiments, the housing cover is cylindrical.

In some embodiments, the housing cover is inflatable, and the apparatus further includes a fluid conduit configured to deliver a fluid into the housing cover such that the fluid inflates the housing cover within the blood vessel.

There is further provided, in accordance with some embodiments of the present disclosure, a system including the apparatus and a processor. The processor is configured to calculate, based on the transduced reflections, a distance between the ablating ultrasound transducer and an inner wall of the blood vessel, and to control the emission of the ablative ultrasound waves in response to the distance.

In some embodiments, the processor is configured to calculate the distance by processing the ultrasound image.

There is further provided, in accordance with some embodiments of the present disclosure, a system including the apparatus and a processor configured to process the ultrasound image so as to identify one or more anatomical structures in the vicinity.

In some embodiments, the processor is configured to process a time series of B-mode images, including the ultrasound image, so as to differentiate between the nerves and other blood vessels.

In some embodiments, the ultrasound image is a B-mode image, and the processor is further configured to process an M-mode image so as to differentiate between the nerves and other blood vessels.

In some embodiments, the ultrasound image is three-dimensional.

In some embodiments, the anatomical structures include the nerves.

In some embodiments, the anatomical structures include one or more other blood vessels.

In some embodiments, the anatomical structures include one or more lymph nodes.

In some embodiments, the anatomical structures include arterial plaque.

In some embodiments, the processor is further configured to mark the anatomical structures in the ultrasound image.

In some embodiments, the processor is further configured to control the emission of the ablative ultrasound waves in response to identifying the anatomical structures.

In some embodiments, the processor is configured to control the emission of the ablative ultrasound waves by controlling a pose of the ablating ultrasound transducer at which the ablating ultrasound transducer emits the ablative ultrasound waves.

In some embodiments, the processor is configured to control the emission of the ablative ultrasound waves by controlling a power of the emission.

In some embodiments, the processor is configured to control the emission of the ablative ultrasound waves by controlling a duration of the emission responsively to a pose of the ablating ultrasound transducer.

There is further provided, in accordance with some embodiments of the present disclosure, a system including the apparatus, further including a temperature sensor disposed within the transducer housing and configured to sense a temperature in the transducer housing, and a processor, configured to modulate an energy of the ablative ultrasound waves in response to the sensed temperature.

There is further provided, in accordance with some embodiments of the present disclosure, a method including inserting a transducer housing, which is coupled distally to a tube and is enclosed by a housing cover, into a blood vessel of a patient, and while moving the transducer housing through the blood vessel, using at least one ablating ultrasound transducer, which is disposed within the transducer housing, emitting ablative ultrasound waves, through the housing cover, at one or more nerves in a vicinity of the blood vessel.

In some embodiments, the housing cover is inflatable, and the method further includes inflating the housing cover within the blood vessel prior to emitting the ablative ultrasound waves.

In some embodiments, emitting the ablative ultrasound waves includes emitting the ablative ultrasound waves while varying a power of the emission.

In some embodiments, emitting the ablative ultrasound waves includes emitting the ablative ultrasound waves while varying a duration of the emission for different poses of the ablating ultrasound transducer.

In some embodiments, the blood vessel is a renal artery.

In some embodiments, moving the transducer housing includes moving the transducer housing through a portion of the renal artery that is downstream from a bifurcation of the renal artery.

In some embodiments, emitting the ablative ultrasound waves includes emitting the ablative ultrasound waves while controlling a pose of the ablating ultrasound transducer.

In some embodiments, controlling the pose includes controlling the pose by, using the tube, rolling the transducer housing.

In some embodiments, controlling the pose includes controlling the pose by, using the tube, moving the transducer housing such that the ablating ultrasound transducer traces a helical path within the blood vessel.

In some embodiments, the method further includes, using at least one imaging ultrasound transducer, which is disposed within the transducer housing, emitting imaging ultrasound waves at the vicinity of the blood vessel such that the imaging ultrasound transducer transduces reflections of the imaging ultrasound waves, and
emitting the ablative ultrasound waves includes emitting the ablative ultrasound waves based on an ultrasound image constructed from the transduced reflections.

In some embodiments, emitting the ablative ultrasound waves includes emitting the ablative ultrasound waves in response to an identification of one or more anatomical structures in the ultrasound image.

In some embodiments, the method further includes, while moving the transducer housing through the blood vessel, using the tube, rolling the transducer housing so as to control an angular range of the imaging ultrasound waves.

There is further provided, in accordance with some embodiments of the present disclosure, a method including inserting, into a blood vessel of a patient, a catheter shaft, a transducer housing, which is coupled distally to a tube disposed within the catheter shaft and houses at least one ultrasound transducer, and a housing cover, which is coupled distally to the catheter shaft and encloses the transducer housing. The method further includes, using the tube, moving the transducer housing through the housing cover, without moving the catheter shaft, while the ultrasound transducer emits ultrasound waves toward a vicinity of the blood vessel.

In some embodiments, the housing cover is inflatable, and the method further includes inflating the housing cover within the blood vessel prior to emitting the ultrasound waves.

In some embodiments, moving the transducer housing through the housing cover includes moving the transducer housing proximally through the housing cover.

In some embodiments, the method further includes, while moving the transducer housing through the housing cover, using the tube, rolling the transducer housing so as to control an angular range of the ultrasound waves.

In some embodiments, the blood vessel is a renal artery.

In some embodiments, moving the transducer housing includes moving the transducer housing while the housing cover is positioned within a portion of the renal artery that is downstream from a bifurcation of the renal artery.

In some embodiments, the ultrasound transducer is an ablating ultrasound transducer, the ultrasound waves are ablative ultrasound waves, and moving the transducer housing includes moving the transducer housing while the ablating ultrasound transducer emits the ablative ultrasound waves at one or more nerves in the vicinity of the blood vessel.

In some embodiments, moving the transducer housing includes moving the transducer housing by initiating an automated process for moving the transducer housing.

In some embodiments, the ultrasound transducer is an imaging ultrasound transducer, the ultrasound waves are imaging ultrasound waves,
moving the transducer housing includes moving the transducer housing while the imaging ultrasound transducer transduces reflections of the imaging ultrasound waves,
the transducer housing further houses at least one ablating ultrasound transducer, and
the method further includes, based on an ultrasound image constructed from the transduced reflections, using the ablating ultrasound transducer, emitting ablative ultrasound waves at one or more nerves in the vicinity.

In some embodiments, moving the transducer housing includes moving the transducer housing in a direction along a path of movement, and emitting the ablative ultrasound waves includes emitting the ablative ultrasound waves while the transducer housing moves in the direction along the path of movement.

In some embodiments, emitting the ablative ultrasound waves includes emitting the ablative ultrasound waves in response to an identification of one or more anatomical structures in the ultrasound image.

In some embodiments, emitting the ablative ultrasound waves includes emitting the ablative ultrasound waves while controlling a pose of the ablating ultrasound transducer.

In some embodiments, controlling the pose includes controlling the pose by, using the tube, rolling the transducer housing.

In some embodiments, controlling the pose includes controlling the pose by, using the tube, moving the transducer housing such that the ablating ultrasound transducer traces a helical path within the blood vessel.

In some embodiments, emitting the ablative ultrasound waves includes emitting the ablative ultrasound waves while varying a power of the emission.

In some embodiments, emitting the ablative ultrasound waves includes emitting the ablative ultrasound waves while varying a duration of the emission for different poses of the ablating ultrasound transducer.

There is further provided, in accordance with some embodiments of the present disclosure, an apparatus including a tube configured for insertion into a blood vessel of a patient, a transducer housing coupled distally to the tube, one or more ultrasound transducers disposed within the transducer housing and configured to emit ultrasound waves toward a vicinity of the blood vessel, and a fluid conduit disposed within the transducer housing at a first side of the ultrasound transducers and configured to deliver a fluid to a distal end of the transducer housing such that the fluid flows proximally, from the distal end of the transducer housing, over the ultrasound transducers at a second side of the ultrasound transducers, which is opposite from the first side.

In some embodiments, the ultrasound transducers include an ablating ultrasound transducer configured to emit ablative ultrasound waves at one or more nerves in the vicinity of the blood vessel.

In some embodiments, the ultrasound transducers further include an imaging ultrasound transducer configured to:
emit imaging ultrasound waves at the vicinity, and
transduce reflections of the imaging ultrasound waves so as to facilitate construction of an ultrasound image for guiding the emission of the ablative ultrasound waves.

In some embodiments, the imaging ultrasound transducer is distal to the ablating ultrasound transducer.

In some embodiments, the apparatus further includes a damping material disposed within the transducer housing,
the imaging ultrasound transducer includes an imaging transducing element disposed within the damping material, and
the fluid conduit passes through the damping material.

In some embodiments, the fluid conduit is configured to deliver the fluid such that the fluid flows into a space between the damping material and a distal inner wall of the transducer housing, and from the space, proximally over the transducer housing.

In some embodiments, the second side is fluidly isolated from the first side, and
the transducer housing contains an air pocket on the first side of the ablating ultrasound transducer, such that the ablative ultrasound waves are concentrated at the second side of the ablative ultrasound transducer.

In some embodiments, the apparatus further includes a housing cover configured to enclose the transducer housing, and the fluid conduit is configured to deliver the fluid such that the fluid flows proximally between the ultrasound transducers and the housing cover.

In some embodiments, the housing cover is cylindrical.

In some embodiments, the housing cover is inflatable, such that the fluid inflates the housing cover as the fluid flows proximally.

In some embodiments, the apparatus further includes a catheter shaft, which contains the tube and is coupled proximally to the housing cover,
and the fluid conduit is configured to deliver the fluid such that the fluid flows proximally through a space between the tube and the catheter shaft.

In some embodiments, the housing cover is coupled distally to the tube, and the fluid conduit is configured to deliver the fluid such that the fluid flows proximally through a lumen of the tube.

In some embodiments, the transducer housing includes a distal aligning element configured to align a guidewire, which passes through the transducer housing, with a roll axis of the transducer housing, such that the guidewire is aligned with the roll axis distally to the transducer housing.

In some embodiments, the apparatus further includes:
a cover surrounding at least part of the aligning element; and
a fluid seal disposed between the cover and the aligning element or distally to the aligning element within the cover.

There is further provided, in accordance with some embodiments of the present disclosure, an apparatus including a tool configured for insertion, over a guidewire, into a body of a patient, and a tube distally coupled to the tool and configured to transfer roll torque to the tool so as to roll the tool about a roll axis. The tool includes a distal aligning element configured to align the guidewire with the roll axis of the tool, such that the guidewire is aligned with the roll axis distally to the tool.

There is further provided, in accordance with some embodiments of the present disclosure, an apparatus including a tube configured for insertion into a blood vessel of a patient, a transducer housing coupled distally to the tube, and an ablating ultrasound transducer disposed within the transducer housing and configured to emit ablative ultrasound waves at one or more nerves in a vicinity of the blood vessel. The ablating ultrasound transducer includes an electroacoustic transducing element and one or more flexible elements that support the electroacoustic transducing element.

In some embodiments, the transducer housing contains an air pocket on a first side of the ablating ultrasound transducer, and the flexible elements isolate a second side of the ablating ultrasound transducer, which is opposite the first side, from the air pocket.

In some embodiments, the flexible elements are configured to facilitate vibration of the electroacoustic transducing element.

In some embodiments, the flexible elements are configured to inhibit short circuiting between two opposing faces of the electroacoustic transducing element.

In some embodiments, the flexible elements include one or more springs.

In some embodiments, the flexible elements include one or more flexible adhesive strips.

In some embodiments, the adhesive strips cover a perimeter of the electroacoustic transducing element.

In some embodiments, the electroacoustic transducing element is configured to vibrate along an axis perpendicular to a plane in which the perimeter of the electroacoustic transducing element lies.

In some embodiments, the adhesive strips include an adhesive selected from the group of adhesives consisting of: an ultraviolet-cured adhesive, a polyurethane adhesive, and a silicon adhesive.

In some embodiments, the apparatus further includes:
a damping material disposed within the transducer housing distally to the ablating ultrasound transducer; and
an imaging transducing element disposed within the damping material,
and the adhesive strips couple the electroacoustic transducing element to the damping material.

In some embodiments, the apparatus further includes an electrical interface disposed proximally, and wiredly connected to, the electroacoustic transducing element, and the adhesive strips couple the electroacoustic transducing element to the electrical interface.

In some embodiments, the adhesive strips couple the electroacoustic transducing element to an inner wall of the transducer housing.

### BRIEF DESCRIPTION OF DRAWINGS

The present disclosure will be more fully understood from the following detailed description of embodiments thereof, taken together with the drawings, in which:

FIG. 1 is a schematic illustration of a denervation system, in accordance with some embodiments of the present disclosure;

FIG. 2 is a schematic illustration of a distal portion of an intracorporeal ultrasound apparatus, in accordance with some embodiments of the present disclosure;

FIG. 3 schematically illustrates an imaging-and-ablating procedure with reference to an example fluoroscopic image, in accordance with some embodiments of the present disclosure;

FIG. 4 is a flow diagram for an example imaging-and-ablating procedure, in accordance with some embodiments of the present disclosure;

FIG. 5A is a schematic illustration of the distal end of an intracorporeal ultrasound apparatus and a cross-section therethrough, in accordance with some embodiments of the present disclosure;

FIG. 5B is a schematic illustration of the distal end of an intracorporeal ultrasound apparatus comprising an inflatable housing cover, in accordance with some embodiments of the present disclosure;

FIG. 6 shows a partly exploded view of an intracorporeal ultrasound apparatus as shown in FIG. 5A, in accordance with some embodiments of the present disclosure;

FIG. 7 shows a longitudinal cross-section through an intracorporeal ultrasound apparatus as shown in FIG. 5A, in accordance with some embodiments of the present disclosure;

FIG. 8A shows a partly exploded view of the distal end of an intracorporeal ultrasound apparatus in accordance with some embodiments of the present disclosure;

FIG. 8B shows a corresponding view of the apparatus in an assembled state, in accordance with some embodiments of the present disclosure;

FIG. 9 is a schematic illustration of the distal end of an intracorporeal ultrasound apparatus, in accordance with some embodiments of the present disclosure;

FIG. 10A shows the distal end of an intracorporeal ultrasound apparatus in accordance with some embodiments of the present disclosure;

FIG. 10B shows a longitudinal cross-section of the distal end of an intracorporeal ultrasound apparatus in accordance with some embodiments of the present disclosure;

FIG. 11 shows another longitudinal cross-section through the distal end of an intracorporeal ultrasound apparatus, in accordance with some embodiments of the present disclosure;

FIG. 12 is a schematic illustration of ultrasound images marked in accordance with some embodiments of the present disclosure; and

FIG. 13 is a schematic illustration of the distal end of an intracorporeal ultrasound apparatus, in accordance with some embodiments of the present disclosure.

### DESCRIPTION OF EMBODIMENTS

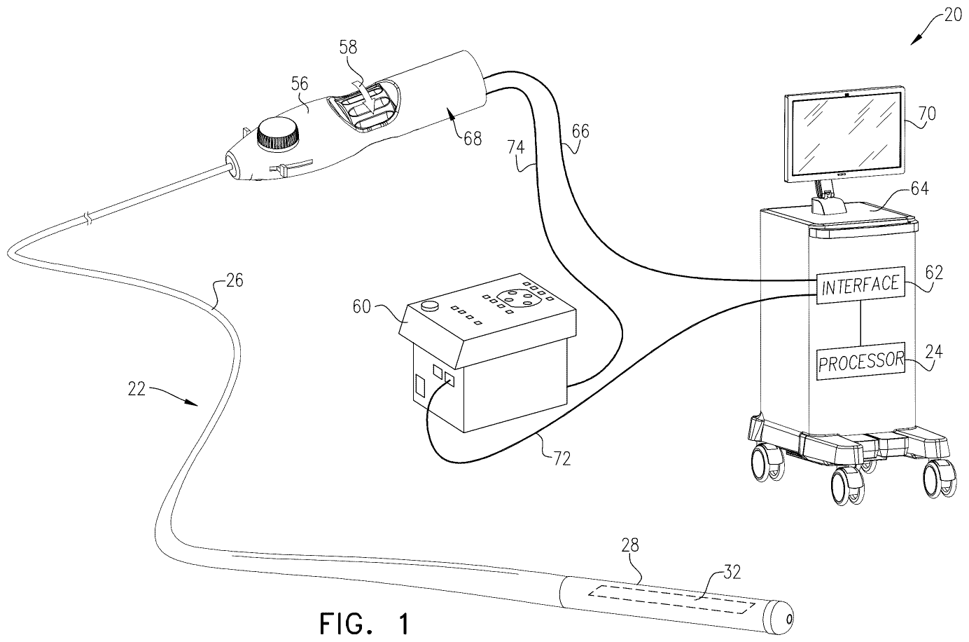

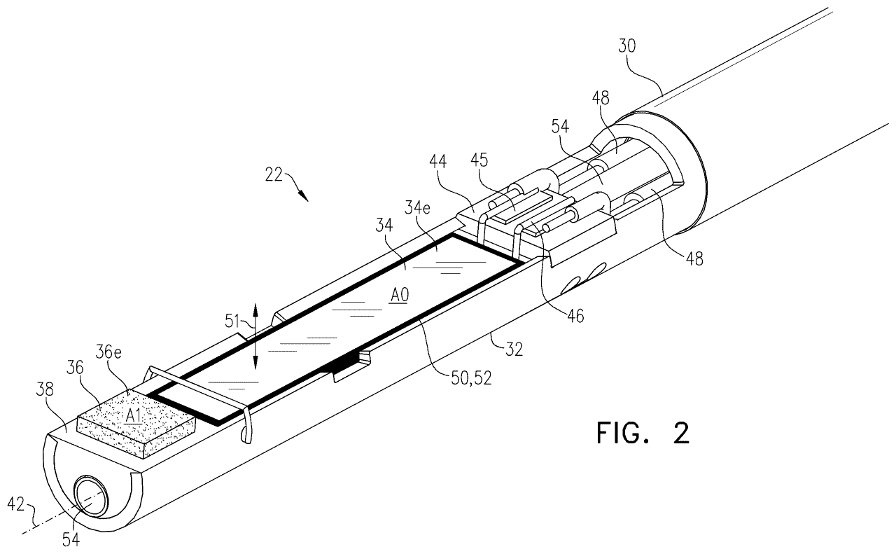

Some embodiments of the present disclosure provide an ultrasonic device, which is shown, for example, in FIGS. 1-2 with reference number 22. The device comprises at least one therapeutic (ablating) transducer 34, which comprises an electroacoustic transducing element, such as a piece of piezoelectric ceramic. The device further comprises a tube 30 configured for insertion into the blood vessel, a transducer housing 32 coupled distally to tube 30, and a housing cover 28, which encloses transducer housing 32 and functions as a low-impedance acoustic window. Transducer 34 is housed in transducer housing 32 and is configured to deliver ablative ultrasound waves, through the acoustic window, to nerves surrounding a blood vessel, such as a renal artery, of a patient.

Typically, it is important to carefully choose the locations at which, and the directions in which, the ablative waves are emitted, to account for “nontarget” anatomical structures, such as blood vessels and lymph nodes, near the renal artery. Likewise, due to the presence of such structures, it may be necessary to carefully control the amount of ablation energy delivered to the vicinity of the artery.

To address these challenges, in some embodiments, the device further comprises at least one imaging transducer 36, which comprises an electroacoustic transducing element, such as a piece of piezoelectric ceramic. During the procedure, the imaging transducer is used to acquire an image of the patient's anatomy in the vicinity of the renal artery. (This image may be a two-dimensional image or a three-dimensional image, which is constructed from an array of two-dimensional images.) The image is analyzed by a healthcare professional, and/or automatically by a computer processor 24 (FIG. 1), so as to identify renal nerves and/or nontarget structures. Based on this identification, the healthcare professional or the processor uses the therapeutic transducer to perform an ablation procedure tailored to the patient's anatomy.

For example, the ablative ultrasound waves may be directed towards any renal nerves identified in the image. Alternatively, the ablative ultrasound waves may be emitted in multiple directions while the device moves through the artery, but the ablation power may be varied, or the emission may be stopped, while the therapeutic transducer faces a nontarget structure. As another example, the speed with which the device is moved through the artery may be varied responsively to the identified nerves or nontarget structures.

Typically, to increase the precision with which the ablation is performed, the transducer housing is configured to restrict the angular range of the ablative ultrasound waves, such that the ablative ultrasound waves do not ablate around the full circumference of the blood vessel simultaneously. For example, in some embodiments, one face of the therapeutic transducing element faces an air pocket, while the other face faces the acoustic window. Thus, advantageously, the ablative ultrasonic waves may be concentrated through the acoustic window. Nonetheless, advantageously, tube 30 is configured to transfer torque to the transducer housing, such that the therapeutic transducer may be rotated so as to ablate in any direction.

In some cases, to reach some of the renal nerves, it may be necessary to position the therapeutic transducer beyond a bifurcation of the renal artery. Hence, in some embodiments, the device is sufficiently small such that the device may be inserted even into the narrower branches of the renal artery. In some embodiments, to facilitate the small size of the device (and also reduce manufacturing costs), the device comprises only a single therapeutic transducer, optionally with a single imaging transducer.

In some embodiments, the therapeutic transducing element is held, within the transducer housing, by a flexible element 50, such as a flexible adhesive strip 52 (FIG. 2). In addition to facilitating vibration of the transducing element, the flexible element may isolate the aforementioned air pocket from the opposite face of the transducing element (from which the ablative waves are emitted), and/or inhibit short circuiting between the two faces of the transducing element.

In some embodiments, the acoustic window is coupled distally to a catheter shaft 26 (FIG. 1). In such embodiments, during the imaging phase of the procedure, the catheter shaft and acoustic window may remain in place while the transducer housing is moved within the acoustic window. Advantageously, this technique may provide greater accuracy in the performance of the subsequent ablation, relative to if the catheter shaft and acoustic window were moved together with the transducer housing. For example, if the acoustic window were withdrawn (i.e., moved upstream) through a segment of the artery during the imaging phase, and it were then decided to ablate along the segment, the acoustic window might need to be repositioned at the downstream end of the segment. However, it might be difficult to perform this repositioning accurately. In contrast, by virtue of the acoustic window being held in place during the imaging phase, no such repositioning is required.

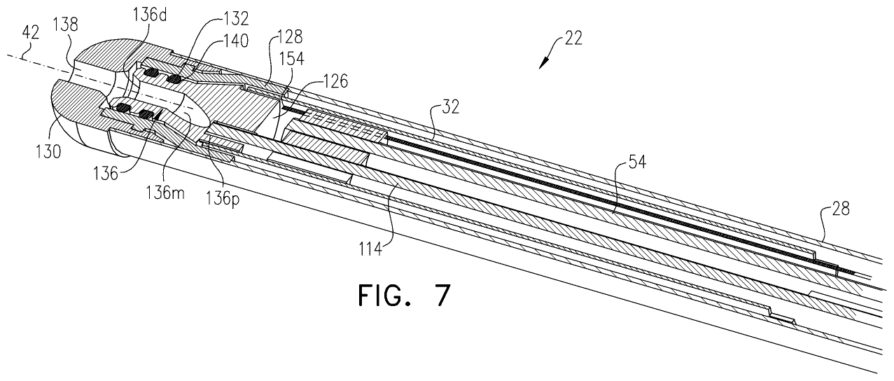

In some embodiments, a fluid is cycled through the device. Advantageously, the fluid may reach the distal end of the device before flowing proximally over the transducer(s), so as to remove any air, including any air pockets at the distal end of the device, that might otherwise impede the ablative and/or imaging ultrasonic waves. Some embodiments provide a fluid seal 132 (FIG. 7) configured to inhibit the escape of fluid into the patient's bloodstream.

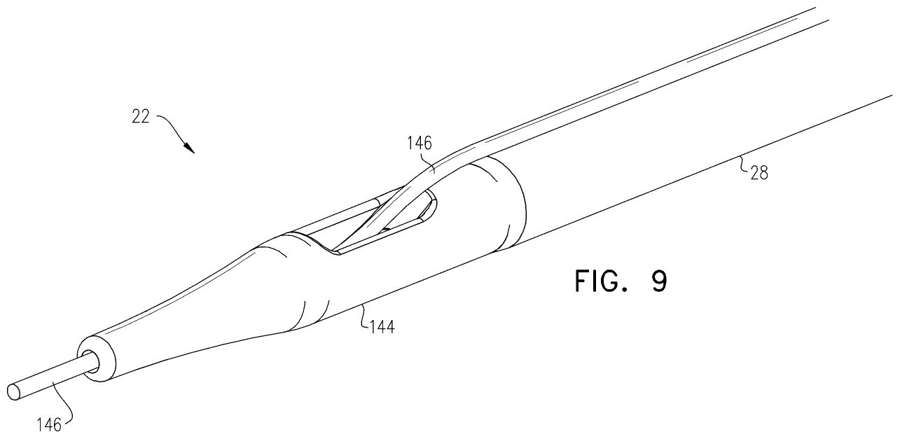

Typically, the device is navigated over a guidewire 146 (FIG. 9). In some embodiments, the guidewire passes through the torque-transfer tube and through an aligning element 126 (FIG. 7) at the distal end of the device. The aligning element aligns the guidewire with the rotational axis of the device, thereby reducing the risk of damaging the artery when the device is rotated. (The aligning element may be integrated with the aforementioned fluid seal.) In other embodiments, the guidewire runs alongside the device, and passes through a rapid exchange tip 144 (FIG. 9) at the distal end of the device.

In addition to renal applications, the apparatuses and methods described herein may be applied to the ablation of other nerves from within other blood vessels. For example, in the treatment of type II diabetes, the hepatic nerves may be ablated from within the hepatic artery. As another example, in the treatment of chronic upper abdominal pain (e.g., as a complication of a disease such as pancreatic cancer or chronic pancreatitis), the celiac plexus nerves may be ablated from within the celiac artery. As yet another example, in the treatment of heart failure, the greater splanchnic nerves may be ablated from within the intercostal vein or intercostal artery.

### System Description

Reference is initially made to FIG. 1, which is a schematic illustration of a denervation system 20, in accordance with some embodiments of the present disclosure.

System 20 comprises an intracorporeal ultrasound apparatus 22. As described in detail below with reference to the subsequent figures, apparatus 22 is configured to ablate nerves, such as renal nerves, in the vicinity of a blood vessel, such as a renal artery, of a patient. In some embodiments, apparatus 22 is further configured to image the vicinity prior to the ablation.

Reference is now additionally made to FIG. 2, which is a schematic illustration of a distal portion of intracorporeal ultrasound apparatus 22, in accordance with some embodiments of the present disclosure.

Apparatus 22 comprises a tube 30, which is configured for insertion into the blood vessel, and a transducer housing 32 coupled distally to the tube. In some embodiments, the length of transducer housing 32 is between 4 and 15 mm, such as between 8 and 12 mm. (It is noted that FIG. 2 omits several features of apparatus 22 such as the distal end of apparatus 22, including the distal end of transducer housing 32.)

Apparatus 22 further comprises at least one ablating ultrasound transducer 34 disposed within transducer housing 32 and configured to emit ablative ultrasound waves at the nerves. Ablating ultrasound transducer 34 comprises an electroacoustic transducing element 34e, which typically comprises a piezoelectric material.

In some embodiments, apparatus 22 further comprises at least one imaging ultrasound transducer 36 disposed within transducer housing 32. Transducer 36 is configured to emit imaging ultrasound waves at the vicinity of the blood vessel, and to transduce reflections of the imaging ultrasound waves so as to facilitate construction of an ultrasound image for guiding the emission of the ablative ultrasound waves. Imaging ultrasound transducer 36 comprises an electroacoustic transducing element 36e, which typically comprises a piezoelectric material.

In general, it is advantageous for the imaging transducer to be as distal as possible within transducer housing 32, so as to limit the distance the transducer housing must be advanced through the blood vessel for imaging purposes. Hence, typically, the imaging ultrasound transducer is distal to the ablating ultrasound transducer.

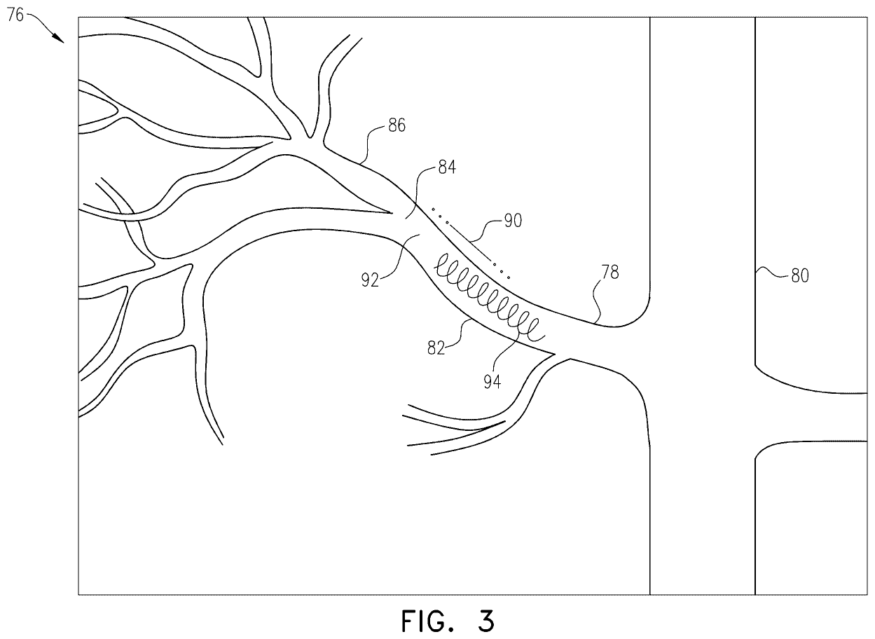

Typically, the ultrasound transducing elements differ from one another with respect to their physical properties, due to the different respective functions of the transducers. For example, whereas imaging transducing element 36e may comprise a soft piezoelectric ceramic, ablating transducing element 34e may comprise a hard piezoelectric ceramic. Alternatively or additionally, the ablating transducing element may have a greater surface area than the imaging transducing element. For example, whereas the surface area A0 of the ablating transducing element may be between 2 and 12 mm², the surface area A1 of the imaging transducing element may be between 0.25 and 1.5 mm², such as 0.5-1 mm². In general, a relatively small surface area A1 may provide greater lateral and elevational resolution, which may help in identifying the nerves in the ultrasound image, as further described below with reference to FIG. 3.

In some embodiments, the transducing elements are configured to vibrate at the same frequency, such as a frequency between 5 and 15 MHz, e.g., 10 MHz. In other embodiments, the transducing elements are configured to vibrate at different respective frequencies. For example, the imaging transducing element may vibrate at a greater frequency (e.g., 20-60 MHz) so as to achieve greater axial resolution, which may help in identifying the nerves in the ultrasound image.

Typically, the ablating transducer is driven with greater power relative to the imaging transducer, such that the ablative ultrasound waves have greater power than do the imaging ultrasound waves. For example, the ablating transducer may be driven with a power of between 1 and 10 W, while the imaging transducer may be driven with a power of 0.1-1 W.

Typically, tube 30 is configured to transfer roll torque to transducer housing 32. Thus, transducer housing 32 may be rolled (i.e., rotated about its roll axis 42 (FIG. 2)) during the imaging and/or ablation phase of the procedure. In addition, tube 30 is configured to transfer linear force to the transducer housing so as to translate (e.g., advance or retract) the transducer housing.

Typically, to reduce manufacturing costs and the overall size of transducer housing 32, apparatus 22 comprises a relatively small number of transducers. For example, apparatus 22 may comprise a single ablating transducer and a single imaging transducer. Nonetheless, advantageously, by rolling the transducer housing, the imaging and/or ablative ultrasound waves may be emitted from the blood vessel in any direction transverse to the blood vessel.

In general, each of the transducing elements may have any suitable shape, such as a rectangular or circular shape.

Typically, imaging transducing element 36e is disposed within a damping material 38 configured to dampen the vibrations of the element so as to facilitate the transduction of the ultrasound reflections. In some embodiments, damping material 38 comprises a combination of tungsten and an adhesive.

Typically, to reduce acoustic impedance, transducer housing 32 does not completely enclose the transducers, as shown in FIG. 2. Alternatively, the transducer housing may be closed.

Typically, apparatus 22 further comprises an electrical interface 44 disposed within the transducer housing and configured to connect the transducing elements, via respective wires 46, to respective conductors of cables 48 running through tube 30. In some embodiments, electrical interface 44 comprises a printed circuit board (PCB) comprising two connecting pads 45, each of which connects a respective one of the transducing elements, and another connecting pad (hidden from view in FIG. 2) that connects the other conductors of cables 48 to ground (typically, the wall of transducer housing 32).

In some embodiments, apparatus 22 further comprises one or more flexible (e.g., elastic or compressible) elements 50 supporting ablating transducing element 34e. Advantageously, flexible elements 50 facilitate the vibration of the ablating transducing element, thereby increasing the efficiency of the ablation. In some embodiments, flexible elements 50 also isolate the two opposite faces of the transducing element from one another, thereby preventing a short circuit between the two faces.

In some such embodiments, flexible elements 50 comprise one or more springs. In other such embodiments, flexible elements 50 comprise one or more flexible adhesive strips 52, which may cover the perimeter of the ablating transducing element. Adhesive strips 52 may comprise, for example, an ultraviolet-cured adhesive, a polyurethane adhesive, or a silicon adhesive. Typically, transducing element 34e vibrates along an axis perpendicular to the plane in which the perimeter of the element lies, as indicated in FIG. 2 by a vibration indicator 51.

For example, in some embodiments, as shown in FIG. 2, ablating transducer 34 is disposed proximally to imaging transducer 36, with damping material 38 interposing between the two transducers. In some such embodiments (not shown), adhesive strips 52 support the ablating transducing element by coupling the element to damping material 38. Alternatively or additionally, electrical interface 44 may be disposed proximally to the ablating transducer, and adhesive strips 52 may couple the ablating transducing element to electrical interface 44. Alternatively or additionally, adhesive strips 52 may couple the ablating transducing element to the inner wall of the transducer housing.

As shown in FIG. 7, in some embodiments, apparatus 22 further comprises a temperature sensor 154 disposed within transducer housing 32 and configured to sense the temperature in the transducer housing. For example, temperature sensor 154 may comprise a coaxial thermocouple at the distal end of a coaxial cable passing through tube 30.

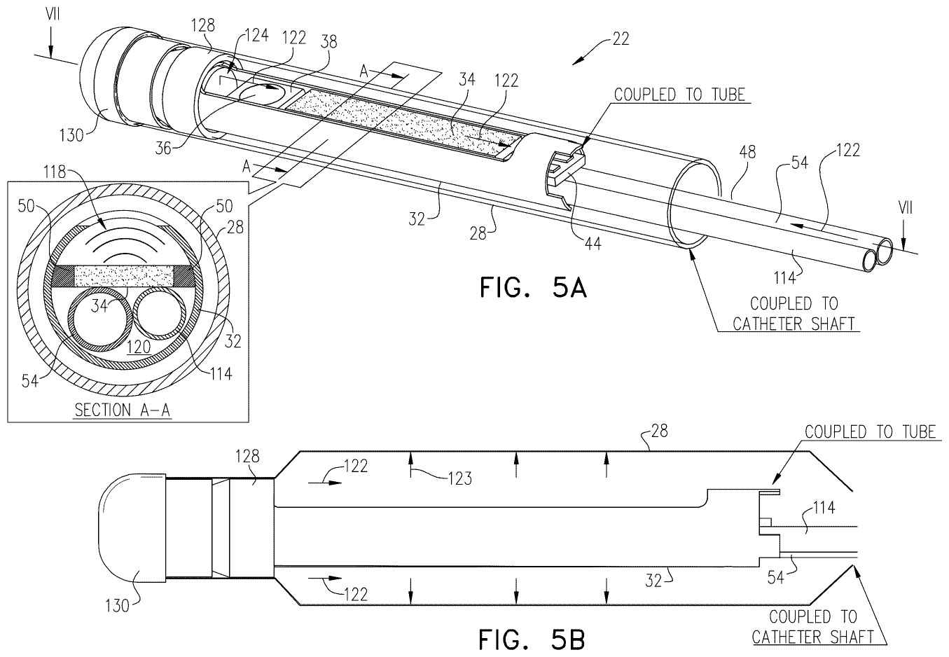

In some embodiments, system 10 further comprises a pump (not shown) configured to pump a fluid through apparatus 22, as further described below with reference to FIG. 5A-B. Apparatus 22 may further comprise a fluid conduit 54 configured to carry the fluid to the distal end of the transducer housing.

As shown in FIG. 1, apparatus 22 typically further comprises a housing cover 28 configured to enclose the transducer housing within the blood vessel, thereby isolating the transducer housing from the surrounding blood. By virtue of housing cover 28 enclosing the transducer housing, any emitted ultrasound waves pass through the housing cover. Typically, housing cover 28 has a relatively small acoustic attenuation, such as an acoustic attenuation less than 3.5 dB/cm at 1 MHz, so as to increase the range of the ultrasound waves. Due to this feature, housing cover 28 may be alternatively referred to as an “acoustic window.” In some embodiments, housing cover 28 is cylindrical.

In some embodiments, the length of housing cover 28 is less than 15 mm. Alternatively or additionally, the diameter of the housing cover may be less than 10 Fr, e.g., less than 7 Fr. Advantageously, these dimensions may increase the maneuverability of the housing cover.

Typically, apparatus 22 further comprises a catheter shaft 26, which contains tube 30.

In some embodiments, catheter shaft 26 is coupled proximally to housing cover 28, i.e., the housing cover is coupled to the distal end of the catheter shaft. In such embodiments, typically, housing cover 28 and catheter shaft 26 do not roll with the transducer housing.

Typically, housing cover 28 has less acoustic impedance than does catheter shaft 26, due to being made of a different material and/or having a thinner wall. For example, the catheter shaft may be made of opaque polyether block amide (PEBA), such as opaque PEBAX®, and the housing cover may be made of optically-transparent PEBA, such as optically-transparent PEBAX®, which has less acoustic impedance than does opaque PEBA.

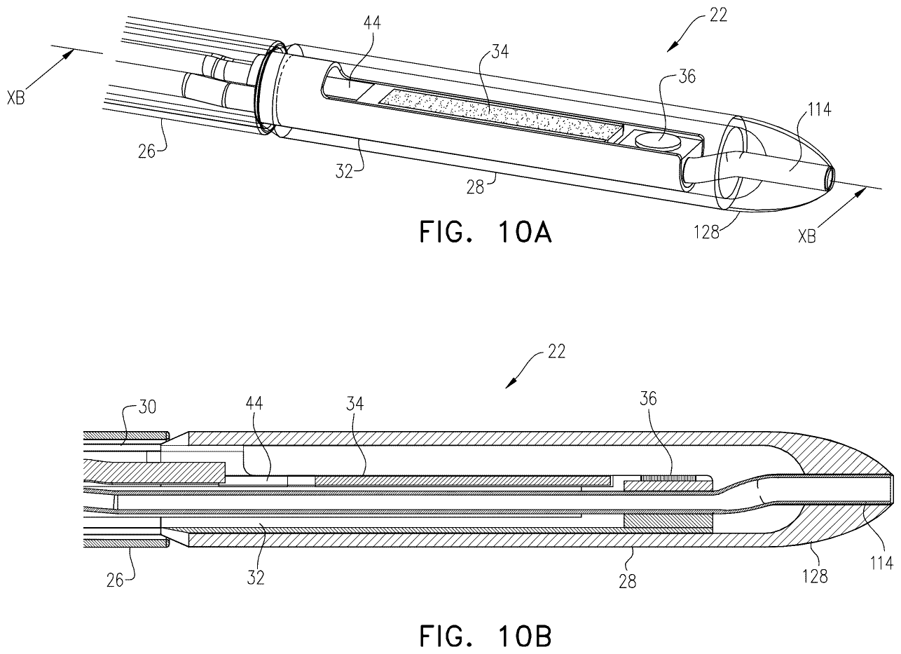

In other embodiments, as further described below with reference to FIGS. 10A-B, housing cover 28 is coupled distally to tube 30 (i.e., the housing cover is coupled to the distal end of the tube), rather than to catheter shaft 26.

Typically, system 20 further comprises a control handle 56 configured to couple proximally to tube 30 and comprising a gear system. During the imaging and/or ablating phase of the procedure, a healthcare professional (e.g., a physician, nurse, or technician) may use the gear system to advance, retract, and/or (as indicated by a roll indicator 58) roll the tube, and hence, the transducer housing.

In some embodiments, the control handle further comprises one or more additional input interfaces, such as buttons. In such embodiments, the healthcare professional may use the additional input interfaces to control the emission of imaging and/or ablating ultrasound waves. Alternatively, the healthcare professional may use a separate input interface, such as a foot pedal, to control the emission of the ultrasound waves.

Typically, control handle 56 is also configured to couple proximally to catheter shaft 26. Using control handle 56, the healthcare professional advances the catheter shaft, together with tube 30, through vasculature of the patient until the catheter shaft is suitably positioned within the blood vessel. Typically, as further described below with reference to FIG. 3, the catheter shaft is advanced through an introducer sheath.

Typically, control handle 56 comprises an electrical interface 68 configured to couple to cables 48 so as to allow the exchange of signals over the cables. Such signals may include signals received from and communicated to imaging transducer 36, signals received from temperature sensor 154 (FIG. 7), and signals communicated to ablating transducer 34.

Typically, control handle 56 further comprises an accelerometer configured to track the location and roll angle of the transducer housing.

In some embodiments, control handle 56 further comprises a memory (not shown), such as an erasable programmable read-only memory (EPROM), configured to store an identifier (e.g., a serial number) of apparatus 22, calibration information (e.g., the exact frequencies at which the transducing elements vibrate), information recorded during previous uses of apparatus 22, and/or any other relevant information.

Typically, system 20 further comprises a processor 24.

In some embodiments, processor 24 is configured to construct an ultrasound image based on signals from imaging transducer 36, which result from the transduction of ultrasound reflections. In some such embodiments, processor 24 is further configured to process the ultrasound image so as to identify one or more anatomical structures in the vicinity of the blood vessel. Alternatively, the processor may identify the anatomical structures by processing the raw signals received from imaging transducer 36, without first constructing an image.

In some embodiments, processor 24 is embodied as a group of multiple processors, which may be cooperatively networked with each other over any suitable wired or wireless communication interface. This group of processors may cooperatively perform the processing functionality described herein in any suitable way. For example, one processor may construct the ultrasound image, and another processor may process the image. Nonetheless, for simplicity, a single processor is assumed in FIG. 1 and in the description below.

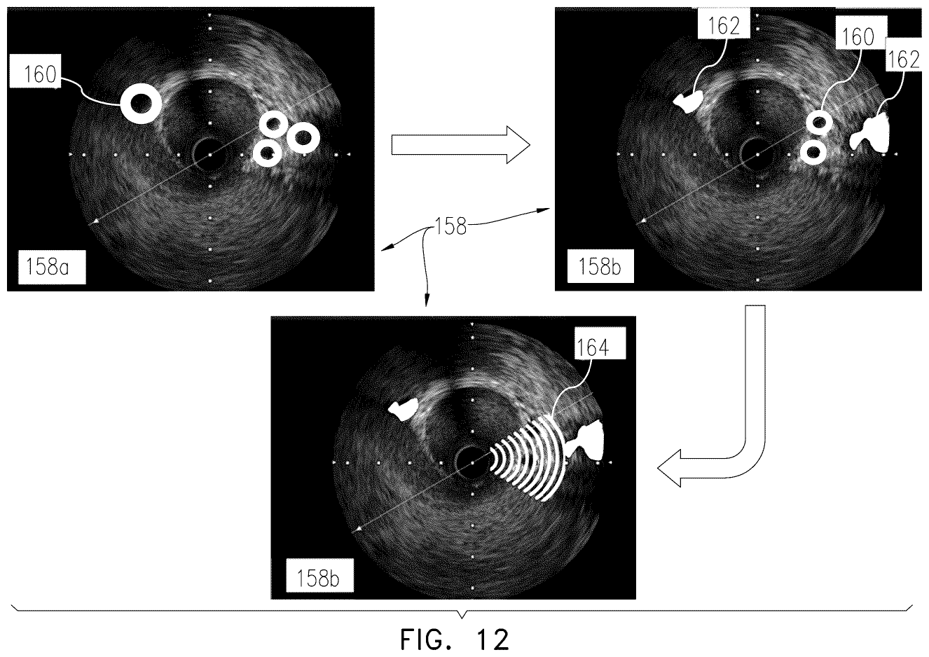

In some embodiments, system 20 further comprises a display 70. In such embodiments, processor 24 may display the acquired ultrasound image on display 70. Optionally, the processor may mark any identified anatomical structures in the ultrasound image, as further described below with reference to FIG. 12.

Alternatively or additionally, as further described below with reference to FIG. 3, processor 24 is configured to control the emission of the ablative ultrasound waves, including, optionally, controlling the locations at which, and the directions in which, these waves are emitted. For example, the processor may perform an automated process for moving the transducer housing such that the desired locations and directions are attained.

In some such embodiments, the aforementioned control functionality is initiated automatically by the processor, in response to identifying anatomical structures in the ultrasound image. Alternatively, this control functionality may be initiated by a user, such the healthcare professional or an assisting technician. In particular, the user may input instructions to the processor via any suitable input interface, such as a touch screen belonging to display 70.

Alternatively or additionally to controlling the emission of the ablative ultrasound waves, the processor may control the emission of the imaging ultrasound waves, including controlling the locations at which, and the directions in which, these waves are emitted.

Typically, processor 24 is connected to interface circuitry 62, which is configured to interface between processor 24 and apparatus 22. For example, interface circuitry 62 may be connected (e.g., via a cable 66 or a wireless connection) to electrical interface 68, such that signals from imaging transducer 36 are passed to interface circuitry 62. Alternatively or additionally, interface circuitry 62 may be connected (e.g., via cable 66 or a wireless connection) to the aforementioned memory in control handle 56, such that the processor may read from and write to the memory. Interface circuitry 62 and processor 24 may be disposed within a console 64.

In some embodiments, interface circuitry 62 comprises a signal generator, an analog noise filter, a signal amplifier, and an analog-to-digital (A/D) converter. Thus, for example, in response to instructions from processor 24, interface circuitry 62 may generate the signals that drive the ultrasound transducers at the distal end of apparatus 22. In addition, after denoising and amplifying the analog signals from the imaging transducer, interface circuitry 62 may digitize the signals and pass the digitized signals to processor 24. Similarly, signals from the temperature sensor and accelerometer may be optionally denoised, digitized, and passed to processor 24. (Alternatively or additionally, processor 24 may perform a digital denoising for any of the aforementioned signals.)

Alternatively to interface circuitry 62, electrical interface 68 may perform the denoising, amplifying, and/or digitizing functionality described above. In such embodiments, electrical interface 68 may interface directly with processor 24.

In some embodiments, system 20 further comprises a motor unit 60. In some such embodiments, motor unit 60 is coupled, via a drive shaft 74, to the gear system in control handle 56. Motor unit 60 is configured to supply mechanical forces to the gear system so as to move tube 30 (and hence, the transducer housing) both linearly and rotationally, independently from catheter shaft 26. In other such embodiments, motor unit 60 is docked within a docking station and is coupled directly both to catheter shaft 26 and to tube 30. The motor unit slides within the docking station so as to move the catheter shaft together with the tube, and supplies rotational force so as to rotate the tube.

Typically, motor unit 60 is connected to interface circuitry 62 via a cable 72, which may be used both for powering the motor unit and for communication. For example, via cable 72, processor 24 may control the motor unit so as to move apparatus 22.

Optionally, motor unit 60 may comprise one or more input interfaces including, for example, a lever, switch, or button. Using the input interfaces, the healthcare professional may drive the motor unit to supply mechanical forces to apparatus 22 so as to move the apparatus. Alternatively or additionally, using the input interfaces, the healthcare professional may provide instructions with respect to the emission of ultrasound waves. These instructions may be communicated from the motor unit, via cable 72 and interface circuitry 62, to processor 24. In response to the instructions, the processor may control the emission of ultrasound waves, e.g., by communicating control signals via cable 66.

The functionality of any of the processors of system 20 may be implemented solely in hardware, e.g., using one or more fixed-function or general-purpose integrated circuits, Application-Specific Integrated Circuits (ASICs), and/or Field-Programmable Gate Arrays (FPGAs). Alternatively, this functionality may be implemented at least partly in software. For example, the processor may be embodied as a programmed processor comprising, for example, a central processing unit (CPU) and/or a Graphics Processing Unit (GPU). Program code, including software programs, and/or data may be loaded for execution and processing by the CPU and/or GPU. The program code and/or data may be downloaded to the processor in electronic form, over a network, for example. Alternatively or additionally, the program code and/or data may be provided and/or stored on non-transitory tangible media, such as magnetic, optical, or electronic memory. Such program code and/or data, when provided to the processor, produce a machine or special-purpose computer, configured to perform the tasks described herein.

### Performing the Procedure

Reference is now additionally made to FIG. 3, which schematically illustrates an imaging-and-ablating procedure with reference to an example fluoroscopic image 76, in accordance with some embodiments of the present disclosure.

Image 76 shows a renal artery 78 branching off from an aorta 80. A main branch 82 of artery 78 bifurcates, at a first bifurcation 84 of the artery, into minor branches 86. Minor branches 86, which may include a superior and/or inferior segmental artery, supply blood to a kidney of the patient. For sake of illustration, FIG. 3 superimposes additional features that are not visible in image 76, such as a renal nerve 90. (Notwithstanding the particular anatomical details shown in FIG. 3, it is noted that the techniques described below with reference to FIG. 3 may also be used for imaging and ablation from within other blood vessels, as described above in the Overview.)

Typically, at the start of the procedure, the healthcare professional navigates an introducer sheath, through aorta 80, to the opening of artery 78. (Optionally, the healthcare professional may insert the introducer sheath into the artery.) Typically, the introducer sheath is navigated over a guidewire 146 (FIG. 9).

This navigation may be performed under the guidance of any suitable imaging modality; for example, the healthcare professional may refer to a fluoroscopic image such as image 76. To facilitate navigation under fluoroscopy, the introducer sheath may comprise a radiopaque material and/or may be coupled to one or more radiopaque markers.

Subsequently, typically via the introducer sheath and over the guidewire, the healthcare professional inserts a distal portion of intracorporeal ultrasound apparatus 22, including transducer housing 32, into artery 78 (in particular, into main branch 82). Any portion of apparatus 22, such as transducer housing 32, may comprise a radiopaque material and/or may be coupled to one or more radiopaque markers, so as to facilitate inserting the apparatus into, and subsequently navigating the apparatus within, within the artery.

Next, the imaging phase of the procedure is performed. In particular, the healthcare professional, or processor 24, moves the transducer housing through artery 78 while imaging transducer 36 emits imaging ultrasound waves at the vicinity of the artery, and transduces reflections of the imaging ultrasound waves. Based on these transduced reflections, processor 24 constructs a two-dimensional image or three-dimensional image (which includes a series of two-dimensional images) of the vicinity. In some embodiments, the processor constructs a three-dimensional anatomical model based on a three-dimensional image.

In some embodiments, during the imaging phase, the transducer housing is moved through a portion of the artery that is downstream from bifurcation 84, such as through a minor branch 86. For example, the transducer housing may reach a location that is at least 2 cm downstream from the bifurcation. Advantageously, as described above with reference to FIG. 1, the relatively small dimensions of housing cover 28 may facilitate the reach of the transducer housing.

Alternatively or additionally, while moving the transducer housing through the artery, using tube 30, the healthcare professional or processor 24 may roll the transducer housing so as to control the angular range of the imaging ultrasound waves. In particular, the transducer housing may be continuously rolled, such that the ultrasound image spans a full 360 degrees around the artery.

Typically, for any segment of the artery for which imaging is desired, the transducer housing is moved upstream through the segment during the imaging. In other words, the healthcare professional first advances the transducer housing to the most downstream portion of the segment, and the transducer housing is then retracted (manually or automatically) while the imaging ultrasound waves are emitted.

Alternatively, the transducer housing may be moved downstream through the segment while the imaging ultrasound waves are emitted.

In some embodiments, the ultrasound image is registered with the image (e.g., fluoroscopic image) used to guide the navigation of apparatus 22. The ultrasound image may then guide subsequent navigation of the apparatus.

Following the imaging phase, the ablating phase of the procedure is performed. In particular, based on the ultrasound image constructed during the imaging phase, using ablating ultrasound transducer 34, ablative ultrasound waves are emitted at one or more nerves 90 within the vicinity of the artery.

More specifically, in some embodiments, the healthcare professional and/or processor 24 identifies anatomical structures of interest in the ultrasound image, and the healthcare professional then performs the ablating phase responsively thereto. (Further details regarding the automatic identification of anatomical structures of interest by processor 24 are provided below with reference to FIG. 12.) Alternatively, as described above with reference to FIG. 1, processor 24 may perform the ablating phase by controlling apparatus 22. As the ablating transducer moves linearly and rotationally within the artery, the power of the ablative waves may be modulated (or “titrated”) based on the distance of the ablating ultrasound transducer from the inner wall of the artery. (In general, for smaller distances, the power is reduced so as to reduce the risk of the inner wall overheating.) Alternatively or additionally, as further described below, the power may be modulated based on the proximity of the ablating transducer to the nerves or to nontarget anatomical structures, such as heat sinks, and/or based on the temperature sensed by temperature sensor 154 (FIG. 7).

Typically, the processor calculates the distance between the ablating transducer and the inner wall of the artery based on the transduced ultrasound reflections. For example, the processor may process the raw transduced signal so as to calculate the distance. (For example, the calculation may be based on the amplitude of the signal and the known speed of the ultrasound wave.) Alternatively, the processor may calculate the distance by processing the ultrasound image using any suitable image-processing techniques (e.g., segmentation).

Typically, the ablation begins at a more downstream location within the artery, and ends at a more upstream location. Alternatively, the ablation may begin upstream and end downstream.

In some embodiments, the identified anatomical structures include the nerves that are to be ablated. Alternatively or additionally, the anatomical structures may include nontarget structures, i.e., structures other than nerves. Nontarget structures may include heat sinks such as blood vessels or lymph nodes, which inhibit the thermal effect required for denervation. Other nontarget structures include arterial plaque or other structures having a relatively large acoustic impedance, and sensitive structures such as the ureter.

The identification of the anatomical structures in the ultrasound image, and/or the calculation of the distance to the inner arterial wall, may facilitate controlling the pose—i.e., the position and angular orientation—at which the ablating ultrasound transducer emits the ablative ultrasound waves. (As described above, the angular orientation may be controlled by rolling the transducer housing.) Alternatively or additionally, the identification of the anatomical structures, and/or the calculation of the distance to the inner arterial wall, may facilitate controlling the power of the emission, and/or the duration of the emission, responsively to the pose of the ablating transducer.

For example, in response to identifying a nerve 90, a “pinpoint” ablation may be performed. In other words, the ablating transducer may be moved to a location 92 opposite the nerve, the transducer housing may be rotated so that the ablating transducer faces the nerve, and the ablative waves may then be emitted.

Alternatively, for example, even if one or more nerves were identified, ablative waves may be emitted while transducer housing 32 is moved through the artery, but the aforementioned parameters of the emission may be varied responsively to the pose of the ablating transducer relative to the identified anatomical structures.

For example, the power of the emission may be reduced (e.g., to zero, i.e., the emission may be stopped) while the ablating transducer faces, and/or is within a threshold distance of, an identified nontarget structure. Alternatively or additionally, the power of the emission may be increased while the ablating transducer faces, and/or is within a threshold distance of, an identified heat sink or structure having greater acoustic impedance, so as to compensate for the effects of these structures. Alternatively or additionally, the power of the emission may be increased while the ablating transducer faces, and/or is within a threshold distance of, a nerve.

Alternatively or additionally, the rate of movement (including linear movement and/or roll) of the transducer housing may be increased while the ablating transducer faces, and/or is within a threshold distance of, an identified nontarget structure, such that less ablative energy is applied to the nontarget structure. Alternatively or additionally, the rate of movement of the transducer housing may be decreased while the ablating transducer faces, and/or is within a threshold distance of, an identified heat sink or structure having greater acoustic impedance, so as to compensate for the effects of these structures. Alternatively or additionally, the rate of movement may be decreased (e.g., to zero, i.e., the transducer housing may be held in position for a predetermined length of time) while the ablating transducer faces, and/or is within a threshold distance of, a nerve.

Alternatively or additionally, the power of the emission may be reduced (e.g., to zero, i.e., the emission may be stopped) while the ablating transducer is within a threshold distance of the arterial wall.

In some such embodiments, using tube 30, transducer housing 32 is moved linearly (i.e., is moved upstream or downstream) and rolled, such that ablating ultrasound transducer 34 traces a helical path 94 within artery 78, as illustrated in FIG. 3. Advantageously, helical path 94 may facilitate greater coverage of the ablative ultrasound waves, relative to other types of paths. The pitch of helical path 94 may be varied responsively to the identified anatomical structures.

In both the imaging and ablating phases, the linear movement of the transducer housing through the artery may be accompanied by linear movement of catheter shaft 26. Alternatively, the transducer housing may be moved linearly without any movement of the catheter shaft.

In some embodiments, during the imaging and/or ablating phase, the roll rate of the transducer housing is 0.1-6 rpm, and/or the rate of linear movement of the transducer housing is 0.1-5 Mm/s.

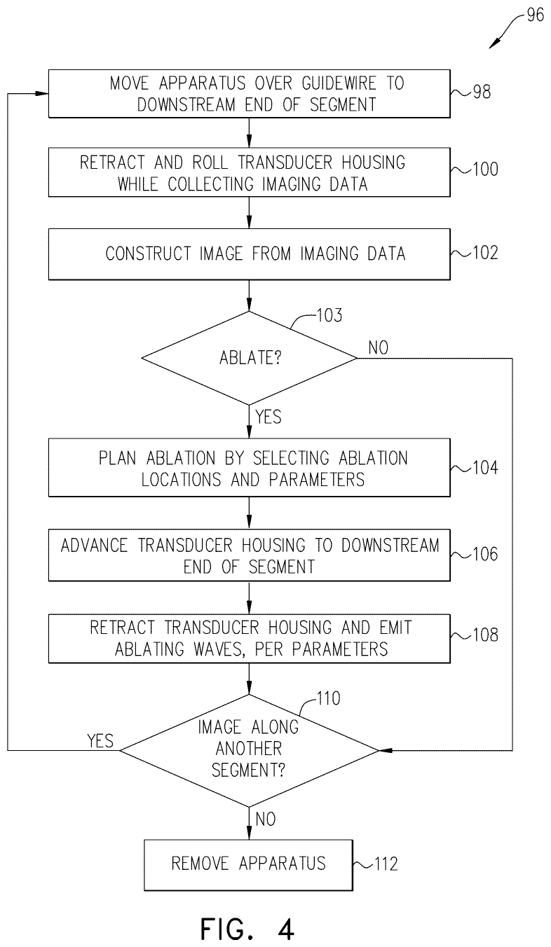

For further details regarding the procedure, reference is now additionally made to FIG. 4, which is a flow diagram for an example imaging-and-ablating procedure 96, in accordance with some embodiments of the present disclosure.

Procedure 96 begins at a positioning step 98, at which apparatus 22 is moved, over a guidewire, to the downstream end of a segment of a blood vessel (e.g., a renal artery). For example, the apparatus may be advanced, over the guidewire, to a location within a minor branch 86 (FIG. 3).

Subsequently, at an imaging step 100, the transducer housing is retracted and rolled while imaging data is collected. (Any of the functions in imaging step 100 may be automatically performed by processor 24.) For example, for arterial applications, at imaging step 100, the transducer housing may be retracted all the way to the entrance to the artery from the aorta, where, it will be recalled, the distal end of the introducer sheath may be located. Thus, for example, the vicinity of the blood vessel may be imaged over a length of the blood vessel spanning 5-80 Mm.

In some embodiments, the catheter shaft—and hence, the housing cover—is retracted while the transducer housing is retracted. In other embodiments, the catheter shaft remains still while the transducer housing is retracted. An advantage of not moving the catheter shaft is that during the subsequent ablating phase, the transducer housing may be more easily moved along the same path of movement, and in the same direction, as during the imaging phase. For example, after retracting the transducer housing (i.e., moving the transducer housing proximally) through the housing cover during the imaging phase, the transducer housing may simply be advanced distally through the housing cover and then retracted again during the ablating phase.

Next, an image and, optionally, a three-dimensional anatomical model is constructed from the imaging data at an image-constructing step 102, thus marking the end of the imaging phase of procedure 96. (In some embodiments, image-constructing step 102 is performed in increments or in real-time, in parallel to the acquisition of the imaging data.)

Following the imaging phase of procedure 96, at a deciding step 103, the healthcare professional decides, based on the image and/or model, whether to ablate at least a portion of the segment, such as along the entire segment, at a specific cross-section of the segment, or at discrete locations along the segment or cross-section. If not, the healthcare professional proceeds to another deciding step 110, described below. Otherwise, at a planning step 104, the healthcare professional plans the ablation by selecting the ablation locations and parameters. As described above, the ablation parameters may include, for example, the locations and powers of the ablative emissions, or the speed at which the transducer housing is moved linearly or rotationally.

The ablation locations are typically selected based on the image and/or model. In some embodiments, the ablation parameters are also selected based on the image and/or model. For example, as described above, planning step 104 may include the identification of anatomical structures of interest in the image, and the selection of the parameters in response thereto.

Subsequently, at an advancing step 106, the transducer housing is advanced to the downstream end of the segment. (In the event that the catheter shaft was retracted at imaging step 100, the catheter shaft is also advanced.) Next, at an ablating step 108, the transducer housing is retracted and ablating waves are emitted, in accordance with the planned parameters. For example, as described above, the ablating waves may be emitted only at select locations, or continuously (e.g., while the ablating transducer follows a helical path) except for when a nontarget structure is within range.

As for imaging step 100, at ablating step 108, the catheter shaft may be retracted together with the transducer housing, or may remain still while the transducer housing is retracted. For example, for some embodiments in which the housing cover is coupled to the catheter shaft, the transducer housing is moved through the housing cover without moving the housing cover. One advantage of the transducer housing moving independently from the housing cover is that it may be easier to configure the system for automatic ablation by the processor, relative to if the housing cover were to move together with the transducer housing.

In some embodiments, planning step 104 and ablating step 108 are performed manually. In other embodiments, one or more of the functions in planning step 104 and/or ablating step 108 are automatically performed by processor 24. For example, at planning step 104, the processor may display the image on display 70, optionally with structures of interest marked. Next, by marking the displayed image (and/or confirming markers placed by the processor), the healthcare professional may select target areas in the vicinity of the blood vessel. Subsequently, at ablating step 108, the processor may automatically direct the ablative ultrasound waves into these areas.

Following ablating step 108, the healthcare professional decides, at deciding step 110, whether to image along another segment, such as a segment immediately upstream from the current segment or a segment of another branch of the blood vessel. If yes, the execution of procedure 96 continues with positioning step 98, in which the apparatus is moved to the downstream end of the new segment. Otherwise, procedure 96 ends, and the apparatus is removed from the body of the patient at an apparatus-removing step 112.

Typically, a significant majority of nerve tissue is within 6 mm of the blood vessel from which the imaging and ablation is performed. Hence, typically, the imaged vicinity of the blood vessel extends less than 10 mm, such as less than 7 mm, from the blood vessel, and most (e.g., more than 80%) of the ablative energy is concentrated in this volume.

In some embodiments, processor 24 is configured to display an alert in response to the temperature sensed by temperature sensor 154 (FIG. 7) exceeding a predefined threshold, such as 50° C. In general, the temperature may exceed the threshold (i.e., the housing cover may overheat) in the event that the housing cover is too close to the wall of the blood vessel, such that there is limited blood flow between the housing cover and the blood vessel wall. In addition, the temperature may exceed the threshold in the event of a problem with the circulation of fluid through the housing cover, or if the ablating transducer becomes less efficient at converting power to acoustic energy.

Alternatively or additionally, the processor may modulate the energy (i.e., the power and/or duration) of the ablative waves in response to the temperature sensed by temperature sensor 154, thereby mitigating any clinical or technical risks. For example, the processor may disable the powering of ablating transducer 34 in response to the temperature exceeding the predefined threshold. Alternatively or additionally, the processor may continually vary the energy so as to keep the temperature within a predefined range.

### Fluid and Guidewire Passage

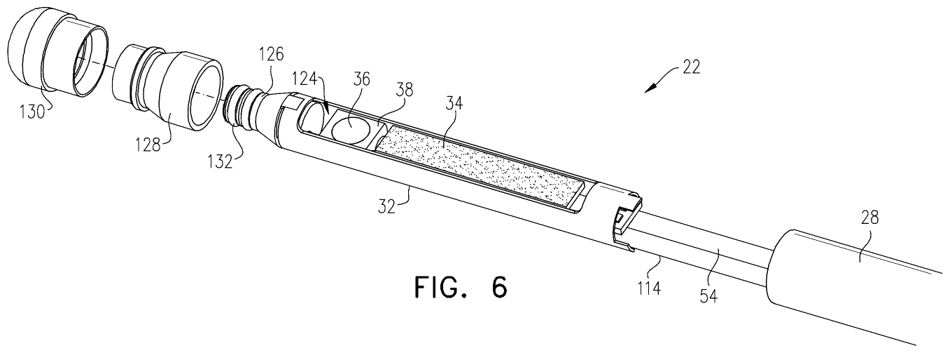

Reference is now made to FIG. 5A, which is a schematic illustration of the distal end of apparatus 22 and a cross-section A-A therethrough, in accordance with some embodiments of the present disclosure. Reference is further made to FIG. 5B, which is a schematic illustration of the distal end of apparatus 22 comprising an inflatable housing cover 28, in accordance with some embodiments of the present disclosure. Reference is also made to FIG. 6, which shows a partly exploded view of apparatus 22 as shown in FIG. 5A.

As described above with reference to FIGS. 1-2, transducer housing 32 is coupled, at its proximal end, to tube 30. As further described above, in some embodiments, housing cover 28 is coupled, at its proximal end, to catheter shaft 26. Transducer housing 32 is configured to move linearly and to roll, within housing cover 28, by virtue of mechanical forces transferred via the tube.

Typically, to increase the precision with which ablative ultrasound waves 118 are directed from apparatus 22, transducer housing 32 is configured to restrict the angular range of the ablative ultrasound waves, such that the ablative ultrasound waves do not ablate around the full circumference of the blood vessel simultaneously.

For example, in some embodiments, transducer housing 32 contains an air pocket 120 on one side of the ablating ultrasound transducer 34. Due to the high acoustic impedance of the air, ablative ultrasound waves 118 are concentrated at the opposing second side of the ablating ultrasound transducer, which is fluidly isolated from the first side (e.g., by flexible elements 50). In such embodiments, typically, fluid conduit 54 is disposed within the transducer housing at the first side of the ablating ultrasound transducer. As indicated by fluid-flow indicators 122 in FIG. 5A, fluid conduit 54 is configured to deliver a fluid, such as distilled water, to the distal end of the transducer housing such that the fluid flows proximally, from the distal end of the transducer housing, over ablating transducer 34 at the second side of the ablating transducer. In particular, the fluid may flow proximally through housing cover 28 such that, due to the exposure of ablating transducer 34 to housing cover 28, the fluid passes between the ablating transducer and the housing cover. Thus, advantageously, the fluid carries away any air in the transducer housing, thereby reducing the acoustic impedance seen by the ablating transducer. Similarly, the fluid may pass between imaging transducer 36 and the housing cover, thereby reducing the acoustic impedance seen by the imaging transducer.

For example, for embodiments in which imaging transducer 36 is distal to ablating transducer 34, fluid conduit 54 may pass through damping material 38 and terminate at the distal side thereof. The fluid may thus flow from the fluid conduit into a space 124 between damping material 38 and the distal inner wall of the transducer housing, and from space 124, flow proximally over transducer housing 32 and through housing cover 28.

Typically, from housing cover 28, the fluid flows proximally into the space between tube 30 and the catheter shaft. The fluid may then continue through this space to the proximal end of apparatus 22, where the fluid may be collected in a container or recirculated through the pump that pumps the fluid.

In other embodiments, the fluid flows distally, into the transducer housing, through the lumen of tube 30, rather than through fluid conduit 54.

In some embodiments, fluid is cycled through the transducer housing continuously while imaging and/or ablation is performed.

In some embodiments, as shown in FIG. 5B, housing cover 28 is inflatable. When inflated, the housing cover can have any suitable shape, such as a cylindrical or an elliptical shape.

Prior to emitting the ablative ultrasound waves (e.g., prior to emitting any ultrasound waves), the housing cover is inflated within the blood vessel, e.g., to a diameter of 3-10 mm. In some embodiments, the housing cover is inflated until the housing cover contacts the wall of the blood vessel. In some such embodiments, the housing cover is shaped to define multiple lobes that contact the wall of the blood vessel while blood flows between the lobes. In other embodiments, the housing cover is inflated to a diameter smaller than that of the blood vessel, such that blood flows between the housing cover and the blood vessel wall.

Typically, the housing cover is inflated, as indicated by inflation indicators 123, by the fluid delivered by fluid conduit 54, as the fluid flows proximally through the housing cover. To deflate the housing cover, the rate of fluid flow is decreased, or the fluid is pumped proximally through the fluid conduit. Alternatively, the fluid for inflating the housing cover is delivered by another fluid conduit.

Advantageously, the inflatable housing cover helps center the ablating transducer within the blood vessel, such that the delivered ablation energy is approximately constant around the circumference of the blood vessel. Furthermore, the increased amount of fluid flowing through the housing cover, relative to a non-inflatable housing cover, carries away an increased amount of heat, such that it may not be necessary to modulate the energy of the ablative waves to avoid overheating.

In some embodiments, the guidewire that guides the passage of the apparatus, as described above with reference to FIG. 3, passes through transducer housing 32, e.g., within a guidewire conduit 114. However, due to design constraints, it may not be possible for the guidewire to be radially centered with respect to the transducer housing, i.e., to be aligned with the roll axis of the transducer housing. Were the guidewire to protrude distally into the blood vessel in this off-center position, the guidewire might cause damage to the blood vessel when the transducer housing is rolled.

Hence, in such embodiments, transducer housing 32 may comprise a distal aligning element 126 (shown in FIG. 6), which may also be referred to as a “centering element,” configured to radially center the guidewire relative to the transducer housing, i.e., to align the guidewire with the roll axis of the transducer housing. Thus, the guidewire may be radially centered, i.e., aligned with the roll axis, distally to the transducer housing.

In some embodiments, aligning element 126 is integral with the more proximal portion of the transducer housing. In other embodiments, aligning element 126 is manufactured separately from, and is then coupled (e.g., glued) to, the more proximal portion of the transducer housing.

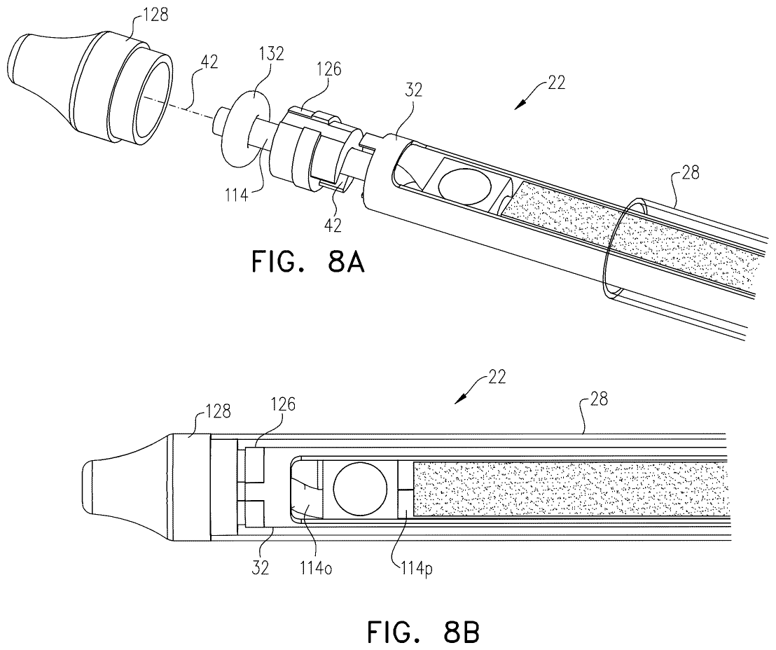

Optionally, apparatus 22 may further comprise a cover 128 surrounding at least part of aligning element 126. In some such embodiments, apparatus 22 further comprises an atraumatic distal cap 130 that fits over cover 128. Housing cover 28 may fit over cover 128 and couple to distal cap 130. Alternatively (e.g., as shown in FIG. 8A-B, described below), cover 128 itself may comprise an atraumatic distal tip, and housing cover 28 may couple to cover 128. By virtue of this coupling, typically, cover 128 and cap 130 do not roll with the transducer housing.

To facilitate the rotation of aligning element 126 within cover 128, a small space between the aligning element and the cover may be required. Consequently, a fluid seal 132 may be disposed between cover 128 and aligning element 126, or distally to the aligning element within the cover, so as to inhibit the fluid flowing through apparatus 22 from escaping through this space into the patient's bloodstream. (Due to the ability of fluid seal 132 to withstand even high fluid pressures, the fluid seal may be referred to as a “dynamic seal.”)

For further details, reference is now made to FIG. 7, which shows a longitudinal cross-section through apparatus 22 as shown in FIG. 5A, in accordance with some embodiments of the present disclosure.

In some embodiments, aligning element 126 is shaped to define a passageway 136 that is at least partly oblique with respect to (i.e., that is at least partly not parallel to) roll axis 42. The aligning element is configured to align the guidewire by virtue of the guidewire passing through passageway 136.

For example, as shown in FIG. 7, passageway 136 may comprise a proximal straight portion 136p, which is parallel to (but not aligned with) roll axis 42, an oblique middle portion 136m, and a straight distal portion 136d, which is aligned with the roll axis. Optionally, guidewire conduit 114 may terminate within, e.g., at the distal end of, proximal straight portion 136p.

For embodiments comprising distal cap 130, the distal cap is shaped to define a bore 138 aligned with the distal end of passageway 136 (and with roll axis 42).

In some embodiments, fluid seal 132 comprises one or more o-rings, which fit around aligning element 126 and thus seal the space between the aligning element and cover 128. Optionally, aligning element 126 may be shaped to define one or more circumferential grooves 140, and the o-rings may sit within grooves 140.

For an alternate embodiment, reference is now made to FIG. 8A, which shows a partly exploded view of the distal end of apparatus 22, and to FIG. 8B, which shows a corresponding view of the apparatus in an assembled state, in accordance with some embodiments of the present disclosure.

In some embodiments, aligning element 126 is shaped to define a straight passageway 142 aligned with roll axis 42, and guidewire conduit 114 passes through passageway 142. Thus, aligning element 126 aligns the guidewire with roll axis 42 by virtue of holding the guidewire conduit in alignment with the roll axis. To facilitate the off-centering of the more proximal portion 114p of the guidewire conduit (per the relevant designs constraints), a portion 114o of the guidewire conduit, which is proximal to aligning element 126, runs obliquely through the transducer housing.

Fluid seal 132 may be disposed between aligning element 126 and cover 128 or, as shown in FIG. 8A, distally to the aligning element.

More generally, aligning element 126 may be used to align a guidewire with the roll axis of any tool configured for insertion, over the guidewire, into the body of a patient. Such a tool may include, for example, an ultrasound-transducer housing (as described at length herein), an optical mirror, a laser-beam emitter, or a bone-burring tool, each of which may be rotated within the body about its roll axis.

For yet another alternative embodiment, reference is now made to FIG. 9, which is a schematic illustration of the distal end of apparatus 22, in accordance with some embodiments of the present disclosure.

In some embodiments, apparatus 22 comprises a rapid exchange tip 144 coupled distally to housing cover 28 and configured for passage of guidewire 146 therethrough. Optionally, rapid exchange tip 144 may be coupled to one or more radiopaque markers, or may comprise a radiopaque material, to facilitate fluoroscopic imaging of the apparatus.

In such embodiments, instead of passing through the tube and transducer housing, guidewire 146 may run alongside the tube (e.g., along the outer wall of catheter shaft 26 (FIG. 1)) and transducer housing, and the transducer housing may roll independently from the guidewire. Thus, there may be no need for guidewire conduit 114 or for aligning element 126 (FIGS. 7-8B). (For example, FIG. 2, which does not show guidewire conduit 114, may correspond an embodiment in which rapid exchange tip 144 is used.)

### Additional Apparatus Designs

Reference is now made to FIG. 10A, which is a schematic illustration of the distal end of apparatus 22, in accordance with some embodiments of the present disclosure. Reference is also made to FIG. 10B, which shows a longitudinal cross-section through apparatus 22 as shown in FIG. 10A, in accordance with some embodiments of the present disclosure.

In some embodiments, housing cover 28 is coupled distally to tube 30. In other words, the distal end of tube 30 is coupled both to the transducer housing and to the housing cover. During the imaging and ablating phases of the procedure, using tube 30, transducer housing 32 may be moved (linearly and/or rotationally) together with housing cover 28.

In such embodiments, the transducers always face the same portion of housing cover 28, which may have a lower acoustic impedance than other portions of the housing cover.

Any of the solutions described above with reference to FIGS. 5-9 may facilitate the passage of the fluid and guidewire. For example, as shown in FIGS. 10A-B, a portion of guidewire conduit 114 may be oblique, typically by virtue of passing through aligning element 126 (FIG. 7). However, given that, in this case, cover 128 rotates with housing cover 28 and aligning element 126, cover 128 may be coupled to the aligning element such that there is no space between these two elements, and hence, fluid seal 132 (FIG. 7) may be omitted. Another difference, relative to FIGS. 5-9, is that the fluid flows proximally through the lumen of tube 30, rather than through the space between tube 30 and catheter shaft 26.

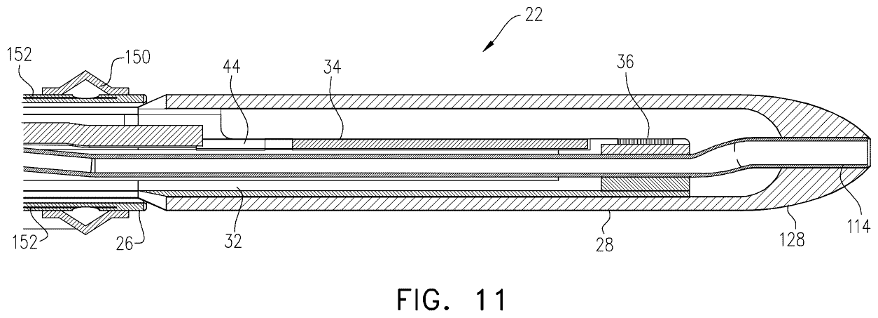

Reference is now made to FIG. 11, which shows another longitudinal cross-section through the distal end of apparatus 22, in accordance with some embodiments of the present disclosure.

FIG. 11 is similar to FIG. 10B, except for the addition of an expandable element 150 coupled to the outer wall of catheter shaft 26. Expandable element 150 is configured to expand within the blood vessel, thereby inhibiting contact between the outer wall and tissue of the blood vessel (and thus enhancing the safety of the procedure). For example, expandable element 150 may comprise a balloon, which is inflated via inflation tubes 152 running within the wall of catheter shaft 26. The transverse cross-sectional shape of the balloon may include one or more gaps, such that the balloon does not impede the flow of blood through the blood vessel; for example, the balloon may comprise four radial fins separated by gaps. Alternatively, for example, expandable element 150 may comprise a stent or another expandable mechanical structure.

As an alternative, expandable element 150 may be coupled to housing cover 28.

For embodiments in which housing cover 28 is coupled to catheter shaft 26 (e.g., as shown in FIGS. 5A-B), expandable element 150 may likewise be coupled to the outer wall of catheter shaft 26 or of housing cover 28.

As noted above, in some embodiments, apparatus 22 comprises at least one ablating transducer without comprising any imaging transducers. In such embodiments, the apparatus may be used for ablating, e.g., as described above with reference to FIGS. 3-4, optionally following the use of a separate apparatus for imaging the vicinity of the blood vessel. For example, after the housing cover and transducer housing are inserted into the blood vessel, the healthcare professional, or the processor, may move the transducer housing through the blood vessel (e.g., through the stationary housing cover) while the ablating transducer emits ablative ultrasound waves, through the housing cover, at one or more nerves in the vicinity of the blood vessel.

Solutions described herein for passage of a fluid and/or guidewire through the apparatus may likewise be applied to embodiments in which the apparatus comprises at least one ablating transducer without comprising any imaging transducers. For example, a fluid conduit disposed within the transducer housing at a first side of the ultrasound transducer may deliver a fluid to the distal end of the transducer housing such that the fluid flows proximally, from the distal end of the transducer housing, over the ultrasound transducer at a second side of the ultrasound transducer. Thus, advantageously, the fluid may remove any air from the transducer housing.

Similarly, flexible elements 50 (FIG. 2) may be used even for those embodiments in which apparatus 22 does not comprise any imaging transducers.

### User Interface

Reference is now made to FIG. 12, which is a schematic illustration of ultrasound images 158 marked in accordance with some embodiments of the present disclosure.

In some embodiments, to help the healthcare professional plan the ablation, processor 24 displays the constructed ultrasound images 158 on display 70 (FIG. 1). In some such embodiments, the healthcare professional may toggle the display between a three-dimensional image and two-dimensional slices from this image. Alternatively or additionally, a three-dimensional image may be displayed simultaneously with one or more two-dimensional images.

In some embodiments—as shown, for example, for a B-mode image 158a in FIG. 12—the processor superimposes markers 160 on the image so as to mark one or more identified anatomical structures of interest in the image, such as nerves or nontarget structures. For example, the processor may draw an ellipse around each of the structures. Typically, the healthcare professional may modify, remove, or add markings for any structures of interest.

To identify the anatomical structures in a B-mode image, the processor may first identify regions of interest in the image using edge detection, segmentation, and/or any other suitable image-processing technique. Subsequently, the processor may identify the anatomical structures based on the shapes and/or gray levels of the regions of interest. For example, regions that are relatively rounded and dark may be identified as nerves or blood vessels. Regions that are relatively bright closer to the transducer and darken moving away from the transducer may be identified as lymph nodes or fibrous sheaths. Alternatively or additionally, the processor may identify an anatomical structure based on its three-dimensional shape, which the processor may compute based on the spatial relationship between regions of interest in an array of B-mode images belonging to a three-dimensional model.

In some embodiments, the processor constructs an M-mode image based on the ultrasound reflections transduced by the imaging transducer, and processes the M-mode image so as to differentiate between nerves and blood vessels, which may appear similar in a B-mode image. For example, an M-mode image 158b in FIG. 12 includes flow indications 162 indicating the flow of blood through blood vessels.

Thus, for example, the processor may initially mark all the structures of interest in B-mode image 158a. Subsequently (e.g., in response to an instruction from the healthcare professional), the processor may construct M-mode image 158b for the same slice of anatomy imaged in B-mode image 158a. In response to identifying flow indications 162, the processor may remove markers 160 from the blood vessels.

Alternatively or additionally, the processor may construct a time series of B-mode images (for the same anatomical slice), which includes B-mode image 158a, based on the transduced ultrasound reflections. Subsequently, the processor may process the time series so as to differentiate between nerves and blood vessels. For example, the processor may identify a blood vessel based on pulsation of the wall of the blood vessel exhibited in the time series. As a specific example, the processor may identify the blood vessel in response to a measure of motion of the wall exceeding a predefined threshold.

In other embodiments, all of the marking is performed manually by the healthcare professional.

In some embodiments, following the healthcare professional's confirmation of the displayed marks, the processor displays suggested ablation parameters and/or automatically performs the ablation.

While the ablation is in progress, the processor may superimpose a wave indicator 164 on the images so as to indicate the direction of ultrasound wave transmission.

In addition to images 158, the processor may display an image used for guiding the procedure, such as image 76 (FIG. 3). Optionally, the healthcare professional may mark the image to indicate sites in the blood vessel at which an ablation was performed. The ablation sites and parameters (e.g., power and duration) and, optionally, the patient's blood pressure measurements over a subsequent period of time, may be stored in a database. Subsequently, in case an additional denervation procedure is required for the same patient, the healthcare professional may refer to the database so as to best plan this procedure. In addition, upon accumulation of sufficient data, from multiple patients, in the database, the data may be analyzed so as to identify sites that are best-suited for ablation.

### Using Optically Pumped Magnetometry

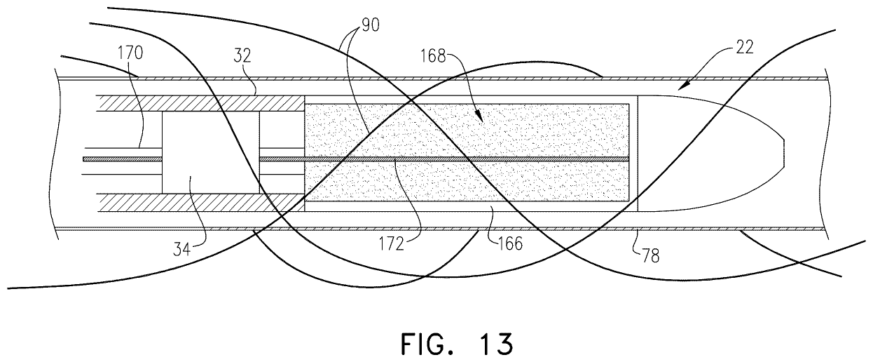

Reference is now made to FIG. 13, which is a schematic illustration of the distal end of apparatus 22, in accordance with some embodiments of the present disclosure.

In some embodiments, transducer housing 32 comprises a vapor cell 166 containing atoms 168 of an alkali metal such as cesium. An optical-fiber cable 170 runs through tube 30 (FIG. 2) to vapor cell 166. Via optical-fiber cable 170, a beam 172 of light (e.g., circularly-polarized laser light) is passed through the vapor cell, and reflections of beam 172 are carried to a photodetector at the proximal end of cable 170. The frequency of the light corresponds to the energy difference between two atomic states of atoms 168.

Due to electric current passing through nerves 90, there is a magnetic field near the nerves. Hence, when the transducer housing is near nerves 90, by virtue of the Zeeman effect, atoms 168 absorb more light from beam 172, such that the photodetector detects less reflection. (The strength of the magnetic field can be assessed based on the degree to which the reflection is reduced.) In response to this decrease in reflection, the healthcare professional may perform an ablation, e.g., by rolling the transducer housing while ablating transducer 34 emits ablating ultrasound waves.

Thus, advantageously, imaging transducer 36 (FIG. 2) may be omitted.

It will be appreciated by persons skilled in the art that the present disclosure is not limited to what has been particularly shown and described hereinabove. Rather, the scope of the present disclosure includes both combinations and subcombinations of the various features described hereinabove, as well as variations and modifications thereof that are not in the prior art, which would occur to persons skilled in the art upon reading the foregoing description. Documents incorporated by reference in the present patent disclosure are to be considered an integral part of the disclosure except that to the extent any terms are defined in these incorporated documents in a manner that conflicts with the definitions made explicitly or implicitly in the present specification, only the definitions in the present specification should be considered.

What is claimed is:

1. A method, comprising:
inserting, into a blood vessel of a patient:
a catheter shaft,
a transducer housing, which is coupled distally to a tube disposed within the catheter shaft and houses at least one ultrasound transducer, and
a housing cover, which is coupled distally to the catheter shaft and encloses the transducer housing; and
using the tube, moving the transducer housing through the housing cover, without moving the catheter shaft, while the ultrasound transducer emits ultrasound waves toward a vicinity of the blood vessel.

2. The method according to claim 1, wherein the housing cover is inflatable, and wherein the method further comprises inflating the housing cover within the blood vessel prior to emitting the ultrasound waves.

3. The method according to claim 1, wherein moving the transducer housing through the housing cover comprises moving the transducer housing proximally through the housing cover.

4. The method according to claim 1, further comprising, while moving the transducer housing through the housing cover, using the tube, rolling the transducer housing so as to control an angular range of the ultrasound waves.

5. The method according to claim 1, wherein the blood vessel is a renal artery.

6. The method according to claim 5, wherein moving the transducer housing comprises moving the transducer housing while the housing cover is positioned within a portion of the renal artery that is downstream from a bifurcation of the renal artery.

7. The method according to claim 1, wherein the ultrasound transducer is an ablating ultrasound transducer, wherein the ultrasound waves are ablative ultrasound waves, and wherein moving the transducer housing comprises moving the transducer housing while the ablating ultrasound transducer emits the ablative ultrasound waves at one or more nerves in the vicinity of the blood vessel.

8. The method according to claim 7, wherein moving the transducer housing comprises moving the transducer housing by initiating an automated process for moving the transducer housing.

9. The method according to claim 1, wherein the ultrasound transducer is an imaging ultrasound transducer,
wherein the ultrasound waves are imaging ultrasound waves,
wherein moving the transducer housing comprises moving the transducer housing while the imaging ultrasound transducer transduces reflections of the imaging ultrasound waves,
wherein the transducer housing further houses at least one ablating ultrasound transducer, and
wherein the method further comprises, based on an ultrasound image constructed from the transduced reflections, using the ablating ultrasound transducer, emitting ablative ultrasound waves at one or more nerves in the vicinity.

10. The method according to claim 9, wherein moving the transducer housing comprises moving the transducer housing in a direction along a path of movement, and wherein emitting the ablative ultrasound waves comprises emitting the ablative ultrasound waves while the transducer housing moves in the direction along the path of movement.

11. The method according to claim 9, wherein emitting the ablative ultrasound waves comprises emitting the ablative ultrasound waves in response to an identification of one or more anatomical structures in the ultrasound image.

12. The method according to claim 9, wherein emitting the ablative ultrasound waves comprises emitting the ablative ultrasound waves while controlling a pose of the ablating ultrasound transducer.

13. The method according to claim 12, wherein controlling the pose comprises controlling the pose by, using the tube, rolling the transducer housing.

14. The method according to claim 12, wherein controlling the pose comprises controlling the pose by, using the tube, moving the transducer housing such that the ablating ultrasound transducer traces a helical path within the blood vessel.

15. The method according to claim 9, wherein emitting the ablative ultrasound waves comprises emitting the ablative ultrasound waves while varying a power of the emission.

16. The method according to claim 9, wherein emitting the ablative ultrasound waves comprises emitting the ablative ultrasound waves while varying a duration of the emission for different poses of the ablating ultrasound transducer.

17. The method according to claim 1, wherein a control handle is configured to couple proximally to the tube and comprises a gear system;
a motor unit is configured to couple to the gear system and to supply mechanical forces to the gear system so as to move the tube; and
an expandable element is coupled to an outer wall of the catheter shaft or of the housing cover and configured to expand within the blood vessel, thereby inhibiting contact between the outer wall and tissue of the blood vessel.

18. The method according to claim 1, wherein a diameter of the housing cover is less than 10 Fr, and the housing cover has a lower acoustic impedance than the catheter shaft.

19. The method according to claim 7, wherein the ablating ultrasound transducer comprises:
an electroacoustic transducing element; and
one or more flexible elements supporting the electroacoustic transducing element, and wherein the flexible elements comprise one or more flexible adhesive strips, which cover a perimeter of the electroacoustic transducing element.

20. The method according to claim 1, wherein the transducer housing comprises a distal aligning element configured to align a guidewire, which passes through the transducer housing, with a roll axis of the transducer housing, such that the guidewire is aligned with the roll axis distally to the transducer housing.
